(19) United States
(12) Patent Application Publication
(10) Pub. No.: US 2013/0245780 A1
(43) Pub. Date: Sep. 19, 2013

(54) Acetabular Cartilage Implant

(71) Applicant: BIOMET MANUFACTURING CORP., Warsaw, IN (US)

(72) Inventors: Jason D. MERIDEW, Warsaw, IN (US); John R. WHITE, Winona Lake, IN (US)

(73) Assignee: BIOMET MANUFACTURING CORP., Warsaw, IN (US)

(21) Appl. No.: 13/891,444

(22) Filed: May 10, 2013

Related U.S. Application Data
(62) Division of application No. 12/915,373, filed on Oct. 29, 2010.

(51) Int. Cl. A61F 2/34

(52) U.S. Cl. CPC A61F 2/34
USPC 623/22.21

(57) ABSTRACT

Disclosed is a system for replacing a portion of cartilage in an acetabulum. A prosthesis can be positioned within an acetabulum to form a portion of the articulation surface within the acetabulum and to articulate with a femoral bone. For example, an acetabular prosthesis can be implanted to articulate with the natural femoral head.

### CROSS-REFERENCE TO RELATED APPLICATIONS

This application is a divisional of U.S. patent application Ser. No. 12/915,373 filed on Oct. 29, 2010. This application includes subject matter similar to that disclosed in U.S. patent application Ser. No. 12/915,366, filed on Oct. 29, 2010, published as U.S. Pat. App. Pub. No. 2012/0109328. The entire disclosures of the above applications are incorporated herein by reference.

### FIELD

An implantable prosthesis to repair or replace a portion of an anatomy, particularly an implantable prosthesis to replace a portion of an articulation region or cartilage of an acetabulum of an anatomy.

### BACKGROUND

This section provides background information related to the present disclosure which is not necessarily prior art.

In an anatomy, such as a human anatomy or other animal anatomy, various bones articulate or move relative to one another. At bone articulation regions, the bones are covered with a material and fluid that ease or cushion articulation. The covering, often cartilage, however can wear or be damaged for various reasons.

One articulation joint in a human anatomy includes the acetabulum which articulates with the femoral head. The joint between the acetabulum and the femoral head is a substantially weight bearing joint as it connects a torso and legs of a human. Accordingly, damage to the acetabulum can cause pain in movement of the human due to decreased cushioning and smoothness of articulation movement due to the damage in the acetabulum. Generally, upon injury or degradation of an acetabulum, the acetabulum can be reamed and replaced with an acetabular prosthesis. The acetabular prosthesis, however, generally replaces the entire acetabular articulation region regardless of the extent of injury or damage to the cartilage in the acetabulum.

### SUMMARY

This section provides a general summary of the disclosure, and is not a comprehensive disclosure of its full scope or all of its features.

According to various embodiments, an acetabular prosthesis for fixing in an acetabulum is disclosed. The acetabular prosthesis can include a member having a concave articulation surface that is operable to articulate with a natural femoral head. The concave surface is formed of pyrolytic carbon to have physical properties substantially similar to the natural femoral head to substantially reduce wear on the natural femoral head compared to a harder articulation surface. According to various embodiments, the entire member is formed of the same pyrolytic carbon or has a substrate substantially completely covered with the pyrolytic carbon. That is at least any portion that will articulate with the femoral head is covered with the pyrolytic carbon. The acetabular prosthesis can further include a bone connection portion extending away from the articulation surface to engage bone near the concave articulation surface. The concave articulation surface is configured to replace a damaged region of acetabular cartilage in an acetabulum.

According to various embodiments, an acetabular prosthesis for fixing in an acetabulum is disclosed. The acetabular prosthesis can include a first region extending away from a first position and having a convex surface on a first side and a second region extending from the first position and having a concave surface on the first side. The prosthesis, therefore, can include both a concave and a convex portion for engagement with a femoral head. The prosthesis can further include a bone connection region on a second side of at least one of the first region and the second region. Generally, the second region is configured to replace a portion of an acetabular cartilage.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is an environmental view of an acetabular prosthesis;

FIG. 2A is a plan view of an acetabular prosthesis;

FIG. 2A′ is a top plan view of the acetabular prosthesis of FIG. 2A;

FIG. 2B is a side plan view of an acetabular prosthesis;

FIG. 2C is a side plan view of an acetabular prosthesis;

FIG. 2D is a side plan view of an acetabular prosthesis;

FIG. 3A is a side plan exploded view of an acetabular prosthesis;

FIG. 3B is a side plan exploded view of an acetabular prosthesis;

FIG. 3C is a side plan view of an acetabular prosthesis;

FIG. 4A is a prospective view of an acetabular prosthesis;

FIG. 4B is a prospective view of an acetabular prosthesis;

FIG. 5A is a side plan view of an acetabular prosthesis;

FIG. 5B is a side plan view of an acetabular prosthesis;

FIG. 6A is a top plan view of an acetabular prosthesis;

FIG. 6B is a top plan view of an acetabular prosthesis;

FIG. 6C is a top plan view of an acetabular prosthesis;

FIG. 6D is a top plan view of an acetabular prosthesis;

FIG. 7 is a environmental view of an acetabular prosthesis;

FIG. 8 is an environmental view of an acetabular and labrum prosthesis;

FIG. 9 is an environmental view of an acetabular and labrum prosthesis;

FIG. 10 is a top prospective view of an acetabular and labrum prosthesis;

FIG. 11 is a cross-sectional and environmental view of the acetabular and labrum prosthesis of FIG. 10; and

FIG. 12 is a cross-sectional and environmental view of an acetabular and labrum prosthesis.

Corresponding reference numerals indicate corresponding parts throughout the several views of the drawings.

### DETAILED DESCRIPTION

Example embodiments will now be described more fully with reference to the accompanying drawings.

With reference to FIG. 1, the human anatomy can include a pelvis 20. The pelvis 20 defines an acetabulum 22 which can include a layer of acetabular cartilage 24 having an acetabular articulation surface 24a that articulates with a femoral head 26. The femoral head 26 can also include femoral cartilage or other surface coverings to articulate with the acetabular cartilage surface 24a. A defect or damaged region 25 may form in the acetabular cartilage 24 when the acetabular cartilage 24 becomes damaged for various reasons, such as injury, disease, natural defect, or the like. Also, the defect or damaged region 25 can be a non-continuous or non-smooth/level region of the acetabular cartilage 24 that causes issues with femoral articulation. Injury to the acetabular cartilage 24 can leave gaps or holes in the acetabular articulation surface 24a that can be repaired to allow for pain free and smooth articulation of the femoral head 26 relative to the acetabulum 22. As discussed above, replacement of the entire acetabulum 22 or even all of the acetabular cartilage 24 with a prosthesis can be performed. However, a point or small prosthesis (also referred to as an implant) or pin 30 can be implanted in the acetabulum 22 and include an articulation surface 32 that can fill the defect to substantially make a continuous acetabular articulation surface 24a to articulate with the natural femoral head 26. The substantially continuous acetabular articulation surface 24a is one that that has fewer or no depressions, holes, bumps, or other defects that can harm or wear unevenly with the femoral head 26. Generally, the articulation surface is generally configured to fit completely within the acetabulum 22 of the pelvis 20 and be, after implantation, continuous with the surrounding and remaining articulation surface of the acetabulum 22. In other words, the prosthesis 30, according to various embodiments, is generally positioned to replace or only replace a portion of the acetabular cartilage 24 when undamaged portions of the cartilage can be maintained or salvaged. Furthermore, the prosthesis 30, according to various embodiments, is positioned substantially completely or entirely within the acetabulum 22.

The surface 32 of the acetabular prosthesis 30 can be formed of a material that allows for smooth articulation of the femoral head 26 without damaging the femoral head 26. For example, pyrolytic carbon can be used to form the surface 32 of the acetabular prosthesis 30 to allow for an appropriate articulation with the femoral head 26. Pyrolytic carbon can be used for articulation of multiple implants, as disclosed in U.S. patent application Ser. No. 12/502,642, filed on Jul. 16, 2009, incorporated herein by reference.

The acetabular prosthesis 30 can further include a bone fixation mechanism, in a bone connection region or portion such as a pin or a tack point 34 that extends from a base or head 36. The pin 34, according to various embodiments including those disclosed herein, can include a geometry and size that is smaller than the dimensions of the head 36. Further, the pin 34 can be contained completely within a bone portion of the pelvis 20 or the acetabulum 22, and generally below the level of any of the acetabular cartilage 24. Thus, as discussed herein, the pin 34, according to various embodiments, can be formed of a material different than the body 36 or the surface 32 and include a size smaller (e.g. substantially smaller in an external dimension, such as about 10-20% of the external size) than the defect 25 in the acetabular cartilage 24.

According to various embodiments, as discussed herein, the acetabular prosthesis 30 can be positioned at appropriate locations within the acetabulum 22 to repair or replace the defect of the acetabular cartilage 24 without replacing all of the acetabular cartilage 24 within the acetabulum 22. Accordingly, as discussed herein, the acetabular prosthesis 30 can be used to repair the damaged articulation region defect 25 that is smaller than the whole acetabular cartilage 24 to substantially make whole the acetabular articulation surface 24a. Generally, the defect 25 may cause the articulation cartilage 24 to have a discontinuous articulation surface. The prosthesis, according to various embodiments, can act to cure or alleviate the defect 25.

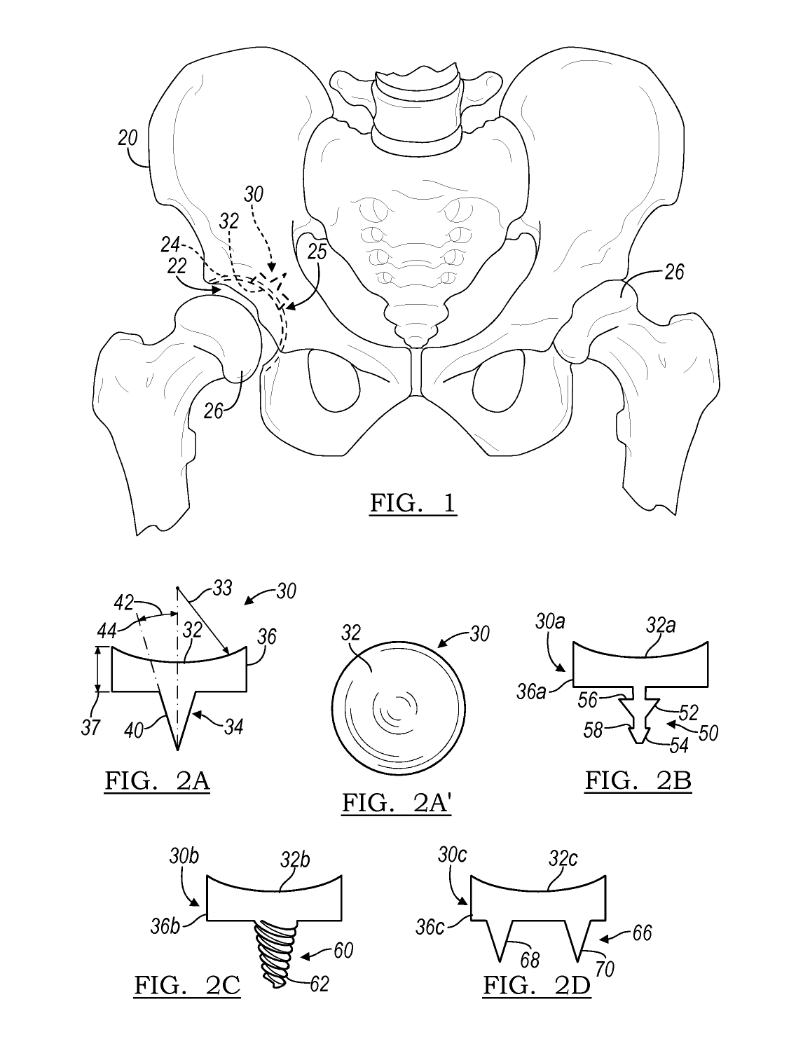

With reference to FIG. 2A, the acetabular prosthesis 30 can include the surface 32 that is formed to substantially match the contour of the acetabular cartilage 24 at the area where it is to be positioned, thus creating at least a local substantially continuous acetabular articulation surface 24a. Generally, the surface 32 of the acetabular prosthesis 30 defines a concave shape. The surface 32, however, can be made to match a specific patient based upon image data acquired of the patient prior to performing a procedure or measurements made while forming a procedure. The acetabular prosthesis 30 can be formed of pyrolytic carbon which can be shaped during a procedure to place the acetabular prosthesis within the acetabulum 22 including shaping and polishing of the surface 32.

The acetabular prosthesis 30 can include the top or body portion 36 from which the bone fixation portion 34 extends. The body portion 36 can be formed to abut or also fit substantially within a blind bore formed in a bone of the pelvis 20 and also to assist in fixation of the acetabular prosthesis 30 to the bone of the pelvis 20. The body 36 can be generally cylindrical or include a taper to assist in fixation or positioning of the prosthesis 30 within the defect 25 of the bone of the pelvis 20.

The body portion 36 can include a height or thickness 37 appropriate to substantially match a thickness of an adjacent portion of acetabular cartilage 24. The height 37 can make sure the articulation surface 32 is continuous and substantially non-interrupted with the adjacent acetabular cartilage 24. Also, as discussed herein the outer perimeter of the body 36 can be substantially round or polygonal. The outer edges of the body can be curved, straight, or have portions that are straight and other portions that are curved.

The bone attachment portion 34 can be used to initially place and hold or to increase fixation of the acetabular prosthesis 30 to the bone of the pelvis 20. As discussed herein, the body portion 36 may also assist in fixation of the acetabular prosthesis 30.

According to various embodiments, all portions of the acetabular prosthesis 30 can be formed of a single material. An exemplary material can include pyrolytic carbon, as discussed above, which includes properties that are substantially similar to bone of a human, including flexibility and hardness. Accordingly, the pyrolytic carbon forming the acetabular prosthesis 30 can articulation with the natural femoral head 26. Harder materials may wear the natural femur and may not be selected. This can also reduce or eliminate the need or selection of replacing the femoral head 26 with a femoral head prosthesis.

The bone fixation section 34 can include a spike or taper defined by a wall 40 of the acetabular prosthesis 30. The wall 40 can be formed at an angle 42 relative to a long axis or central axis 44 of the bone attachment portion 34. The central axis 44 can also be a central axis for the surface 32 of the acetabular prosthesis. The angle 42 can be any appropriate angle selected for fixation of the acetabular prosthesis into the pelvis 20. For example, the angle 42 can be about 1 degree to about 45 degrees to allow for at least initial fixation of the acetabular prosthesis 30 to the bone of the pelvis. The angle 42 can also be about 5 degrees to about 30 degrees, and about 7 degrees to about 25 degrees.

The articulation surface 32, as illustrated in FIG. 2A, can be substantially concave. The concave surface of the articulation surface 32 can be provided to substantially match or allow for smooth articulation relative to the acetabular cartilage 24 and have a selected radius 48. Thus, the articulation surface can be formed to have an appropriate radius 48. Also, a plurality of prosthesis can be provided in a kit each with different radii. Generally, the radius 48 is selected to coincide or be continuous with the curve of the adjacent acetabular cartilage 24.

The articular surface 32 can also be substantial polished to provide a smooth surface for articulation with a femoral head 26. According to various embodiments, as discussed above, the surface 32 can be formed of pyrolytic carbon. Generally, the acetabular implant 30 will be a xeno graft. Thus, the material forming the acetabular prosthesis 30 is generally not naturally formed from the anatomy from a similar donor anatomy. Nevertheless, pyrolytic carbon can provide an articulation surface that substantially imitates the natural anatomy. Alternatively, the articulator surface 32 can be substantially highly polished biocompatible materials including ultra high molecular polyethylene and metals or metal alloys (e.g. stainless steel, cobalt chromium alloys, titanium alloys, and pure titanium).

As illustrated in FIG. 2A, the acetabular prosthesis 30 can be formed as a substantially single-piece. Accordingly, the articular surface 32, the body 36, and the bone attachment portion 34 are a single-piece that are formed unitarily, such as through injection molding, casting, or other formation techniques. It will also be understood that the acetabular prosthesis 30 can be formed as multiple pieces and integrally fixed together into one-piece prior to implantation into the pelvis 20. For example, the body 36 can be formed and the bone attachment portion 34 can be fixed to the body by using appropriate techniques, such as fusing, adhesives, welding, and the like. Such modular initial construction can still allow the acetabular prosthesis 30 to be implanted as an integral one-piece prosthesis, even if the prosthesis is not unitarily formed as a single-piece.

With reference to FIG. 2B, an acetabular prosthesis 30a is illustrated. The acetabular prosthesis 30a can include portions that are similar to the acetabular prosthesis 30 including a body 36a and an articulation surface 32a. A bone attachment portion 50, however, can include one or more barbs, such as a first barb 52 and a second barb 54. The second barb 54 can be a terminal barb that also includes a distal tip that can be shaped to allow for impaction into the pelvis 20. The barbs 52, 54 can include tapered or conical surfaces that terminate in upper flat surfaces 56 and 58, respectively. The flat surfaces 56, 58 can resist withdrawal of the acetabular prosthesis 30a from the pelvis 20 after implantation of the acetabular prosthesis 38. It will be understood that a plurality of the barbs or a single barb can be provided to assist in holding the acetabular prosthesis 30a into the pelvis 20. The taper angle of the barbs 52, 54 can be similar to the angle 42 of the bone connection portion 34, but could be provided for ease of driving the acetabular prosthesis into the bone due to the positioning of the anti-withdrawal flats 56, 58.

Again, the body 36a and the bone attachment portion 50 can be unitarily formed as a single piece or can be formed separately and later connected to form the single piece acetabular prosthesis 30a. Additionally, the articulation surface 32a can be similar to the articular surface 32 of the acetabular prosthesis 30. Additionally, the acetabular prosthesis 30a can be formed of materials that are substantially identical to the acetabular prosthesis 30 to allow for articulation with the femoral head 26 of a natural anatomy and to replace the damaged portion 25 of the acetabular cartilage 24.

According to various embodiments, an acetabular prosthesis 30b is illustrated in FIG. 2C. The acetabular prosthesis 30b can include portions similar to the acetabular prosthesis 30 including a body 36b and an articulation surface 32b. The acetabular prosthesis 30b, however, can include a bone attachment portion 60 that defines one or more twists or threads 62. The threads 62 can allow for twisting or driving the acetabular prosthesis 30b into the bone of the pelvis 20. It will be understood that the body 36b can include a substantially non-circular shape to allow for engagement with a tool. For example, the exterior surface of the body 36b can be substantially hexagonal to engage a driving tool. Alternately, the thread 62 can be used to resist withdrawal after the acetabular prosthesis is impacted axially into the bone of the pelvis 20. Therefore, the bone fixation portion 60 need not require that the prosthesis 30b be twisted into the bone, but can be axially impacted, such as with a hammer.

The dimensions and configurations and materials of the acetabular prosthesis 30b can be substantially similar to those of the acetabular prosthesis 30. Accordingly, the acetabular prosthesis 30b can be formed of pyrolytic carbon or other appropriate materials. Additionally, the body 36b can be formed unitarily as a single member with the bone attachment portion 60 or can be formed in two pieces later fixed together using appropriate fusing techniques. Additionally, the thread 62 can be formed in a mold during formation of the acetabular prosthesis 30b or can be worked from the bone attachment portion 60, such as with milling or cutting.

An acetabular prosthesis 30c, illustrated in FIG. 2D, can include portions that are similar to the acetabular prosthesis 30 illustrated in FIG. 2A. Thus, the acetabular prosthesis 30c can include an articulation surface 32c and a body 36c. The acetabular prosthesis 30c, however, can include a bone attachment portion 66 that includes one or a plurality of projections 68, 70, according to various configurations. As exemplarily illustrated, the projection 68, 70 can be a substantially similar to the bone engaging portion 34 of the acetabular prosthesis 30. It will be understood, however, that barbs can be provided on the bone attachment portion 66, threads can be provided on the bone attachment portion 66, or other removal restrictive mechanisms can be formed on the bone attachment portion 66. It will be understood, however, that a plurality of projections can be provided in the bone attachment portion extending from the body 36c to engage the bone of the pelvis 20.

The acetabular prosthesis 30c can be formed of appropriate materials including those discussed above regarding the acetabular prosthesis 30. For example, the acetabular prosthesis 30 can be formed entirely of pyrolytic carbon to articulate with the natural femoral head 26. Additionally, the bone attachment portion 66 can be formed unitarily as a single piece with the body 36c or can be formed separately and later fused to the body 36c.

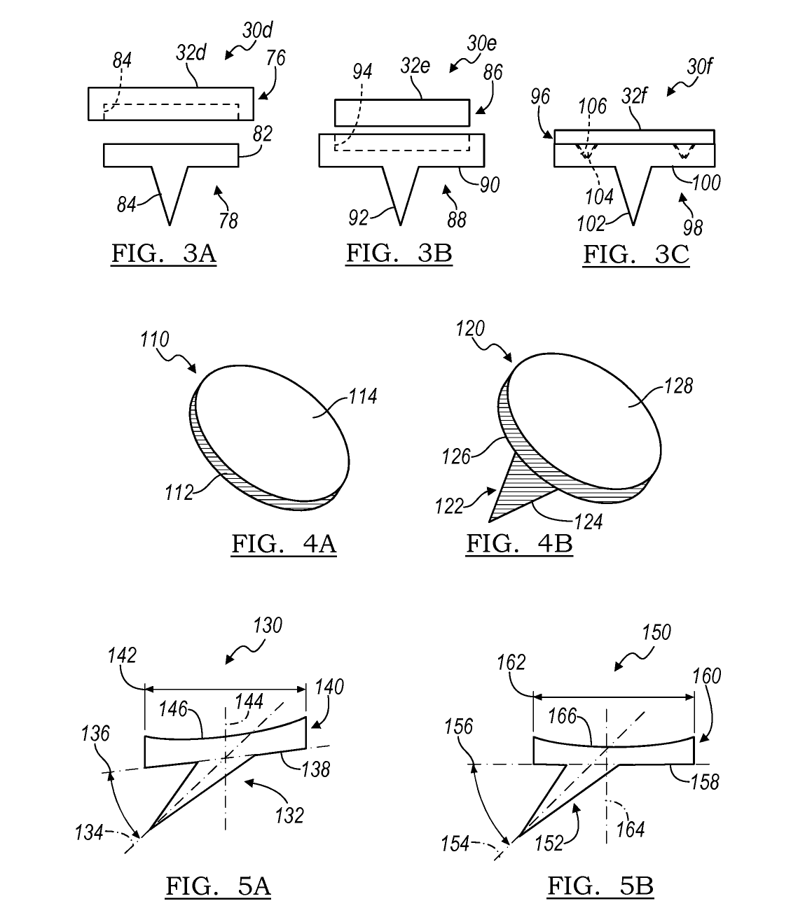

With reference to FIG. 3A, an acetabular prosthesis 30d is illustrated. The acetabular prosthesis 30d can include an articulating surface 32d similar to the articulation surface 32 discussed in relation to the acetabular prosthesis 30. The articulation surface 32d, however, can be defined by a first member 76 that is formed to fit onto a second member 78. The second member 78 can define the bone connection portion 80, which can be any appropriate bone connection portion, including those discussed above, such as the tapered spike 40 illustrated relative to the acetabular implant 30. The second member 78 can also form a body portion 82 that is received or fits into a recess 84 formed in the first member 76. The recess 84 can be substantially complementary to the body connection portion 82 to allow for appropriate fit.

The two members, including the first member 76 and the second member 78 can be formed separately to allow for the use of a different material for the two members. For example, the first member or articulating member 76 can be formed of a pyrolytic carbon, similar to that discussed above, to allow for articulation with the natural femoral head 26. The properties of the pyrolytic carbon can allow for the articulation of the femoral head 26 with the acetabular implant 30d with low or no wear on the natural femoral head 26.

The first member 76 can then be connected to the second member 78 with any appropriate mechanism, such as an interference fit, adhesives, snap fit, screw fit, taper connection, or the like. The connection of the first member 76 to the second member 78 allows the bone connection mechanism 80 to be formed using the properties of the second material of the second member 78 that may not be possible with the material properties of the first member 76, such as pyrolytic carbon. For example, a thread or fixation configuration can be formed in the bone contact or fixation portion 80 that can provide additional holding power. For example, certain configurations (e.g. thickness, width) of the second member when formed of a metal may not be appropriate for fixation to bone when the second member is formed of the pyrolytic carbon material of the first member 76. Although the combination of the first member 76 and the second member 78 can provide similar benefits of the substantially single or uniformly formed material members of the acetabular prostheses discussed and illustrated in FIGS. 2A-2D.

With reference to FIG. 3B, an acetabular implant 30e is illustrated. The acetabular implant 30e can include an articulation surface 32e similar to the articulation surface 32 illustrated and referenced to the acetabular implant in FIG. 32A. The acetabular implant 30e, however, can include a first member 86 and a second member 88. The second member 88 can include a body portion 90 and a bone connection portion 92. The second member 88 can also include a recess 94 that is configured and shaped to receive the first member 86 and the first member 86 can be connected to the second member 88 in a manner similar to that of the first member 76 to the second member 78. This can allow the first member 86 to be formed of a separate material or different material than the second member 88. The materials of the first member 86 and the second member 88 can be similar to the materials of the first member 76 and the second member 78 of the acetabular implant 30d illustrated in FIG. 3A. Accordingly, the properties of the bone connection portion 92 can be provided based upon the material properties of the second member 88, which may not be possible with the material properties of the first member 86. Again, the first member 86 can be formed of a material to articulate with the natural femoral head 26, such as pyrolytic carbon. Also, the bone connection portion 92 can be formed in any appropriate configuration, including those discussed above, such as interference fit, or a point or spike similar to the taper portion 40 illustrated in FIG. 2A.

With reference to FIG. 3C, an acetabular prosthesis 30f is illustrated. The acetabular prosthesis 30f can include a first member 96 and a second member 98. The second member 98 can include a body portion 100 and a bone connection portion 102. The body portion 100 can have a perimeter dimension that is substantially equivalent to a perimeter dimension of the first member 96. The second member 98, however, can be formed of a different material in the first member 96. The first member 96 can then be adhered to the second member 98 with any appropriate mechanism, such as adhesives, welding, and the like. The perimeter dimensions that are substantially identical and complementary, allow for the first member 96 to substantially cover the second member 98, but allows for a complete support of the first member 96 with the second member 98. The second member 98 can be formed of a different material than the first member 96 and they can be adhered together using appropriate mechanisms, such as adhesives, welding, internal attachment mechanisms (e.g. projections 104 from the first member 96 that engages, in an interference fit with recesses 106 in the second member 98).

Accordingly, as illustrated in FIGS. 3A-3C, it will be understood that the acetabular prostheses 30d-30f illustrate that a pyrolytic carbon articulation portion can be provided to articulate with the natural femoral head 26 while allowing for a second portion to be provided to fit within the acetabulum 22 of the patient. This allows the bone connection portions 80, 82, 102 to be formed of appropriate materials and selected mechanisms to fixedly hold the respective acetabular implants 30d-3f within the acetabulum 22. The respective first members 76, 86, 96 can be fixed to the respective second members 78, 88, and 98 with appropriate mechanisms including interferences fits, adhesives (e.g. bone cement), and other fixation mechanisms.

With reference to FIG. 4A, an acetabular prosthesis 110 is illustrated. The acetabular implant 110 can be formed to include a porous metal bone fixation portion 112 and an articulation portion or surface 114. The articulation surface 114 can be formed of appropriate materials, such as highly polished metals, including cobalt chrome alloys, or non-metal materials such as pyrolytic carbon. As discussed above, pyrolytic carbon can be used for articulation substantially with the natural femoral head 26 when the acetabular implant is positioned in the acetabulum 22.

The bone fixation portion 112 that can be formed of a porous metal that can allow for bone ingrowth for fixation of the acetabular implant 110. Porous metal portions can include Regenerex™ sold by Biomet, Inc. The porous metal bone fixation portion 112 can define substantially an entire surface of the acetabular implant 110 for fixation in the acetabular 22. The articulation surface 114 can be similar to the articulation surface 32, as discussed in relation to the acetabular implant 30 in FIG. 2A. Accordingly, the articulation surface 114 can be substantially concave to fill a void or defect in the acetabular cartilage 24. The pyrolytic carbon or other material used to form the articulation surface 114 can be mated to the porous metal portion 112. The articulation surface 114 can be mated to the porous metal portion 112 by interdigitation of the articulation surface into the porous metal portion 112, adhesives, or similar fixation mechanisms. Thus, the acetabular implant 110 can be integrated as one-piece for implantation into the acetabulum during an operative procedure. The articulation surface 114 can be substantially polished to allow for smooth articulation of the femoral head 26.

With reference to FIG. 4B, an acetabular prosthesis 120 is illustrated that includes a bone attachment portion 122 that can include a bone piercing region 124 (e.g. spikes, barbs, etc.) and a bone contacting region 126. The bone piercing and bone contacting regions 124, 126 can be formed of a porous metal, such as a Regenerex™ sold by Biomet, Inc. The porous metal can allow for boney ingrowth to form a selected and long term fixation of the acetabular prosthesis 120 relative to the acetabulum 22.

An articulation surface 128 can be formed on the acetabular prosthesis 120 and can be formed of materials for articulation with the femoral head 26. For example, the articulation surface 128 can be metal alloys, such as cobalt chromium alloys, that are highly polished. Alternatively, the articulation surface 128 can be pyrolytic carbon to allow for articulation with the natural femoral head 26 and substantial reduction or elimination of possible wear of the femoral head 26.

The acetabular implant 110, 120 illustrated in FIGS. 4A and 4B can allow for fixation to the acetabulum 22 in the pelvis 20 using the porous ingrowth properties of the porous metals. Porous metals allow for ingrowth throughout a volume of the bone fixation regions 112, 122 for long term fixation of the acetabular implants 110, 120 relative to the pelvis 20. In addition, the acetabular implants 110, 120 can be formed to fill the defect 25 in the acetabular cartilage 24 that does not fill the entire acetabulum 22 of the patient.

With reference to FIG. 5A, an acetabular implant 130 is illustrated. The acetabular implant 130 can be any of the acetabular implants illustrated and discussed in FIGS. 2A-4B. The acetabular prosthesis 130, however, can include a bone fixation region 132 that has a central or long axis 134 that forms an angle 136 relative to a body or contacting surface 138 of a body portion 140 of the acetabular prosthesis 130. The body portion 140 can include a dimension, such as an edge to edge dimension 142 and a central axis 144 that generally divides the dimension 142 in half. The acetabular prosthesis 130 can also include an articulation surface 146 to articulate with the femoral head 26.

The angle 136 can allow for a selected position of the acetabular prosthesis 130 in the acetabulum and for selected fixation properties. For example, the acetabular prosthesis 130 can be driven at an angle into the acetabulum 22 to allow for fixation of the acetabular prosthesis 130 within the acetabulum 22 once the femoral head 26 is positioned back into the acetabulum 22 and articulates with the acetabular prosthesis 130. The angle 136 can allow for a pressure or force to be applied to the acetabular prosthesis 130 generally along the axis 134 after the femoral head 26 is positioned back into the acetabulum 22. In one example, the defect 25 may be in a position in the acetabulum 22 where the angle 136 of the bone fixation region 132 can be aligned with a natural axis of force from the femoral head 26 once the femur is placed back into the acetabulum 22. The axis of force of the femur may be the vertical axis or inferior-to-superior axis of force through the femoral head 26 to the acetabulum 22. The angle 136 can be selected and formed in the acetabular prosthesis 130 according to the location of the defect 25 and the anatomy of the patient. Accordingly, it will be understood that the angle 136 can be formed based upon a particular anatomy of the patient and the position of the defect 25 relative to the acetabulum 22.

With reference to FIG. 5B, an acetabular prosthesis 150 is illustrated. The acetabular prosthesis 150 can include a bone connection region 152 that substantially defines a long axis or axis 154 that can form an angle 156 with a bottom surface 158 of a body portion 160 of the acetabular prosthesis 150. A cross dimension 162 can define a distance from edge to edge of the acetabular prosthesis 150 and an axis 164 can generally divide the distance 162 in half. As illustrated in FIG. 5B, the axis 154 of the bone fixation region 152 need not intersect the dividing axis 164 at the bottom surface 158 of the body region 160. Accordingly, the bone fixation region 152 may be offset from a center of the acetabular prosthesis 150 for fixation into the acetabulum 22.

The positioning of the bone fixation region 152 can allow for positioning of the acetabular prosthesis 150 into the acetabulum and for maintaining fixation of the acetabular prosthesis 150 and into the acetabulum 22. Again, based upon the anatomy of the patient, the femoral head 26 articulates with an articulation surface 166 and the pressure of the femoral head 26 against the articulation surface 166 can assist in holding the acetabular prosthesis 150 in the acetabulum 22. The offset distance or position of intersection of the axis 154 of the bone connection region 152 with the dividing axis 164 can be selected based upon the position of the acetabular prosthesis 150 within the acetabulum 22 and the remaining anatomy, such as the configuration and placement of the femoral head 26 within the acetabulum 22. The selected position can assist in fixation and maintaining a fixation position of the acetabular prosthesis 150 once positioned in the acetabulum 22. The angle 156, amount and position of the offset, and other features or positions of the bone fixation portion 152 can be selected for applying a force along the axis 154 similar to the fixation types as discussed in relation to the acetabular prosthesis 130.

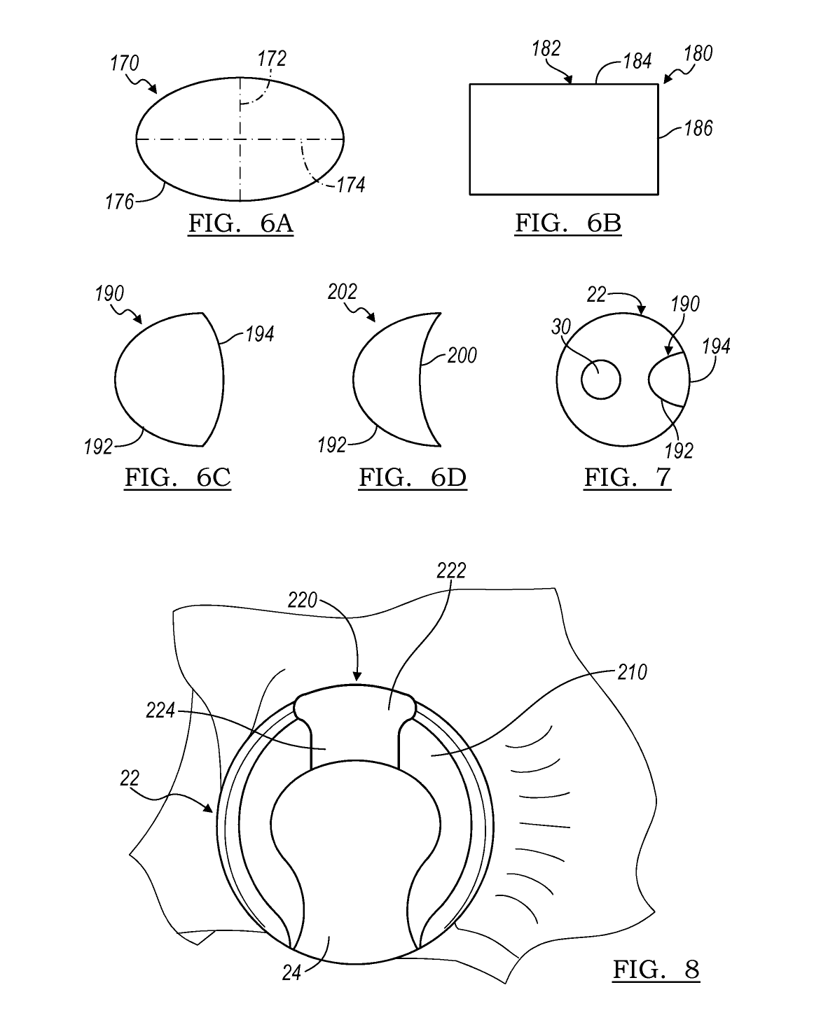

With reference to FIGS. 6A-6D, acetabular prosthesis, according to various embodiments can be included in selected shapes. As illustrated in FIGS. 6A-6D, a plurality of shapes can be provided, and those exemplarily illustrated are provided for illustration purposes only. Accordingly, an acetabular prosthesis can be included in appropriate shapes including substantially circular shapes illustrated above and the irregular or regular geometric shapes illustrated in FIGS. 6A-6D.

In particular, an acetabular prosthesis 170, illustrated in FIG. 6A, can include an oval shape. Accordingly, the acetabular prosthesis 170 can include a minor axis 172 and a major axis 174. The minor and major axes 172, 174 can be selected based upon the size of the defect 25 in the acetabulum 22. It will be understood that the shape and size of the acetabular prosthesis can be selected preoperatively using various techniques, such as exploratory surgery or imaging, or can be provided in a kit of a plurality of sizes and dimensions to be selected by a user during an operative procedure. Nevertheless, an oval exterior perimeter 176 can be provided to fill a selected defect shape.

With reference to FIG. 6B, an acetabular prosthesis 180 is illustrated to have rectangular perimeter 182. The perimeter 182 can include a long side 184 and a short side 186. Although FIG. 6B, illustrates a substantially regular rectangle, it will be understood that a trapezoid or other irregular shapes can also be provided. Similarly, a square can be provided with equal length sides.

With reference to FIG. 6C, a partial circle or oval and convex acetabular prosthesis 190 is illustrated. The acetabular prosthesis 190 can include a first portion 192 that has a small radius and a second portion 194 that has a large radius. The large radius portion 194 can be substantially convex in the acetabular prosthesis 190. It will be understood, however, as illustrated in FIG. 6D, that a second portion 200 having a large radius can form a substantially concave region or portion of an acetabular prosthesis 202. The acetabular prosthesis 202 can include the first portion 192 that is substantially similar in radius to the first portion 192 of the acetabular prosthesis 190. Thus, a concave or a convex configuration can be formed for the acetabular prosthesis as selected.

As illustrated in FIG. 7, an irregularly shaped acetabular prosthesis can be used to fill the acetabulum 22 in various irregular shapes and/or substantially along an edge of the acetabulum 22. For example, as illustrated in FIG. 7, the acetabular prosthesis 190 can have the first portion 192 that extends into the acetabulum and the second portion 194 that is generally positioned along the edge of the acetabulum 22. Thus, the acetabular prosthesis, according to various embodiments, can be positioned on an edge of the acetabulum 22 and have a non-uniform shape to conform with the acetabulum 22.

It will be understood that the acetabular prosthesis can be provided as one member or as a plurality within the acetabulum 22 of the patient. Thus, one or more of the acetabular prostheses can be provided in the acetabulum 22 to fill one or a plurality of defects 25. For example, as illustrated in FIG. 7, a round acetabular prosthesis, such as the acetabular prosthesis 30, can also be provided within the acetabulum 22. Thus, a plurality of acetabular prosthesis can be provided in a single acetabulum 22 to fill multiple or a large defects without requiring replacement of the entire acetabulum, such as with an acetabular implant.

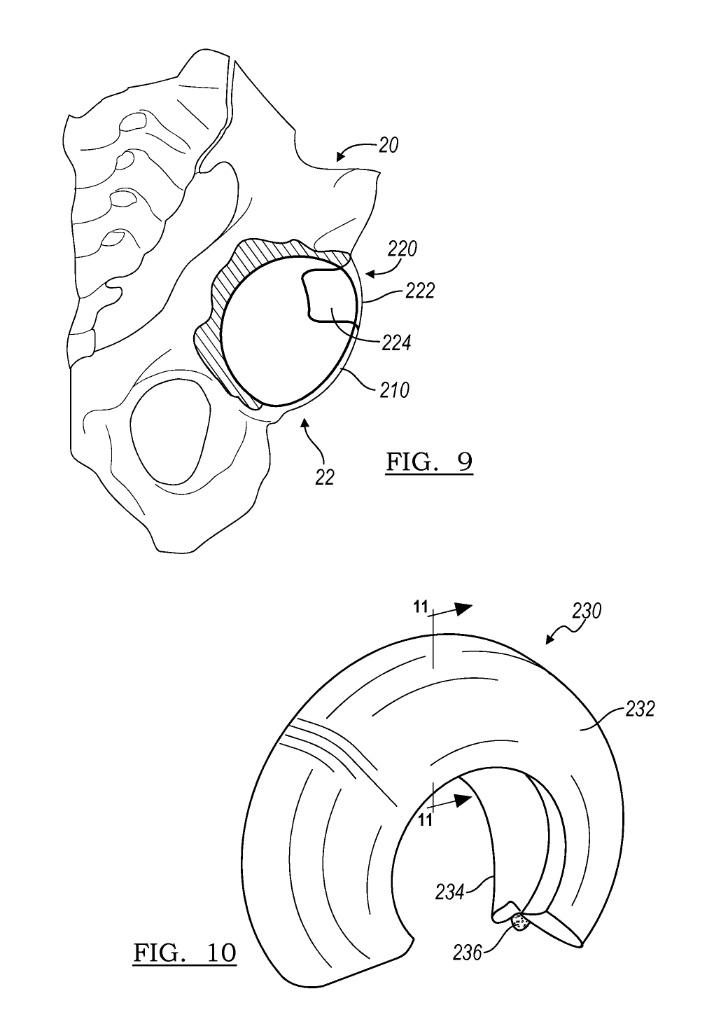

With reference to FIGS. 8 and 9, the acetabulum 22, as generally understood by one skilled in the art, includes a labrum 210 that generally surrounds the socket and cartilage region 24. The labrum 210 can be damaged through injury, use, age, and the like and a labrum prosthesis 220 can be used to replace a portion or all of the labrum 210. The labrum prosthesis 220 can include a portion that defines a labrum portion 222 that extends out of the cartilage region 24 of the acetabulum 22. Labrum implants can include those disclosed in U.S. patent application Ser. No. 12/915,366, filed on Oct. 29, 2010, published as U.S. Pat. App. Pub. No. 2012/0109328, incorporated herein by reference. The labrum prosthesis 220 can also include an articular cartilage region 224 extending into the region that generally articulates with the femoral head 26.

The labrum prosthesis 220 can includes two regions where the labrum region 222 is convex and the acetabular cartilage region 224 is concave. The two differently shaped regions can allow the labrum implant 210 to be positioned to replace both a damaged region of the labrum and an adjoining damaged region of the acetabular cartilage region 24. Thus, the labrum implant 210 can be provided to replace all appropriate damaged regions of the acetabulum 22 for articulation with the femoral head 26.

The labrum prosthesis 220 can be formed of appropriate material such as pyrolytic carbon, including those discussed above. The pyrolytic carbon can be provided to articulate with the natural femoral head 26 with only minimal wear due to the properties of the pyrolytic carbon. Additionally, however, the labrum prosthesis 220 can be formed of other appropriate materials, including metals and metal alloys such as cobalt metal alloys. Metal alloys can be highly polished to articulate smoothly with natural anatomical portions, such as the femoral head 26.

With reference to FIGS. 10 and 11, a labrum acetabular prosthesis 230 is illustrated. The labrum acetabular prosthesis 230 is exemplarily illustrated and substantially operable to replace an entire labrum portion of an acetabular region of the patient. It will be understood, however, that the labrum implant 230 can be sized to replace only a portion of the labrum and the acetabulum 22, similar to the dimension illustrated in FIGS. 8 and 9. Nevertheless, the labrum prosthesis 230 can include a labrum portion 232 and an acetabular cartilage region 234. The labrum acetabular prosthesis 230 can be fixed to the acetabulum 22 of the patient with a porous metal fixation portion 236.

As illustrated in FIG. 11, the acetabular cartilage prosthesis portion 234 extends into the acetabulum 22 of the patient and replaces a portion of the natural cartilage 24 in the acetabulum 22. The porous metal fixation portion 236 can be fitted against bone relative to the acetabulum 22 to allow for bone ingrowth and fixation of the labrum prosthesis 230. The labrum portion 232 can then extend above or out of the acetabulum 22 to contact the femoral head 26 in a substantially natural limiting matter. Accordingly, the acetabular and labrum prosthesis 230 can include both the concave portion defined by the acetabular cartilage replacement portion 234, and the convex portion defined by the labrum replacement portion 232. Again, it will be understood that the labrum portion 232 can be formed of pyrolytic carbon as can the acetabular cartilage replacement portion 234. The porous metal portion 236 can be fixed to the pyrolytic carbon portion in any appropriate manner, such as with adhesives, welding, or interdigitation by the pyrolytic carbon portions.

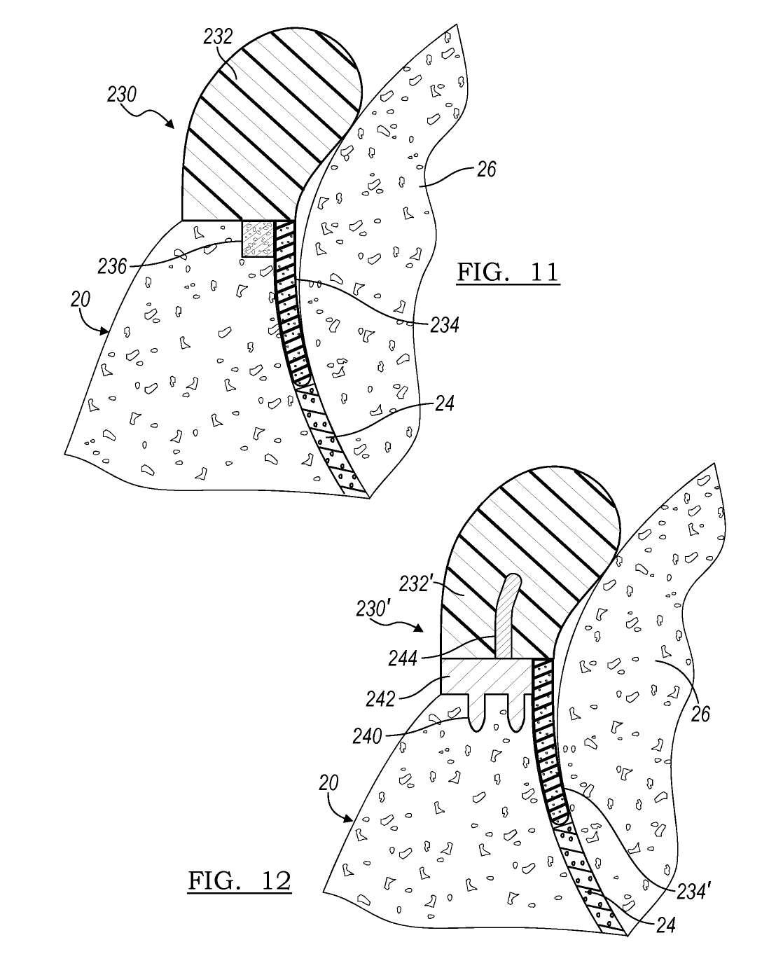

With reference to FIG. 12, the labrum and acetabular prosthesis can be fixed to the pelvis 20 with appropriate bone fixation portions, including a spike or projection 240. The spike 240 can extend from a base plate 242 or from a bottom of a labrum and acetabular prosthesis 230. Other fixation mechanisms can also be used, such as separate screws, barbs, rivets, etc. The base plate 242 can include a second projection or labrum engaging projection 244 to assist in fixation and stiffen of the labrum portion 232 of the labrum and acetabulum prosthesis 230. The bone connection projections can be driven directly into the bone of the pelvis 20 or can be fitted into pre-drilled or pilot holes in the pelvis 20. It will be understood that additional fixation mechanisms can be provided, such as adhesives (e.g. bone cement) to allow for initial or permanent fixation of the acetabulum and labor prosthesis 230.

It will be understood that the acetabular prosthesis or the acetabular labrum prosthesis can be provided in appropriate dimensions based upon defects in the patient to be replaced. Additionally, a kit including two or more of the different types of acetabular prostheses, sizes of the acetabular prostheses, or shapes of the acetabular prostheses, or labrum acetabular prosthesis can also be provided. Accordingly, during an operative procedure, a user, such as a surgeon can determine or select an appropriately sized prosthesis for filling a determined or formed defect in the acetabulum 22 of the patient. Alternatively, or in addition to providing a plurality of prostheses in a kit, preoperative planning can be used to identify the appropriate size, shape, and type of prosthesis that can be selected for a selected procedure. The preoperative planning can include imaging or exploratory surgery of the patient to investigate the acetabulum 22 of the patient to identify the defect 25 in the acetabulum 22 to replace with an appropriate prosthesis. Accordingly, it will be understood that one or more of the prostheses can be provided during a single operative procedure and one or more of the prostheses can be selected to be put into the acetabulum 22 to treat one or more defects in the acetabulum 22.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. An acetabular prosthesis for fixing within an acetabulum, comprising:
a single piece body member having a concave articulation surface that is configured to articulate with a natural femoral head, at least a portion of the body formed of a selected material having physical properties substantially similar to the natural femoral head; and
a bone connection portion having a dimension configured relative to a selected region and having a bone engaging surface extending from the single piece body opposite the concave articulation surface to engage bone;
wherein the concave articulation surface is sized and configured to replace the selected region of acetabular cartilage in the acetabulum and placed in the selected region to be substantially continuous with adjacent acetabular cartilage in the acetabulum.

2. The acetabular prosthesis of claim 1, wherein the bone connection portion includes at least one bone engaging projection extending from a bone contacting surface of the body;
wherein the concave articulation surface is opposite the bone contacting surface;
wherein the bone contacting surface is configured to contact the acetabulum and support the body and the concave articulation surface.

3. The acetabular prosthesis of claim 2, wherein the bone contacting surface of the body is formed of a porous metal material operable to allow boney ingrowth from the acetabulum to hold the body in a selected location.

4. The acetabular prosthesis of claim 3, wherein the at least one projection extending from the bone contacting surface is also formed of the porous metal to allow bony ingrowth to hold the body at a selected location in the acetabulum.

5. The acetabular prosthesis of claim 2, wherein the at least one projection defines a projection long axis that is substantially coaxial with a central body axis that extends substantially perpendicular to the bone contacting surface and substantially through a center of the single piece body;
where an exterior surface of the at least one projection extends at an angle relative to the projection long axis of the at least one projection and intersects the projection long axis of the at least one projection at a distance from the bone contacting surface.

6. The acetabular prosthesis of claim 2, wherein the at least one projection includes at least one barb having a holding surface extending generally perpendicular to a projection long axis of the at least one projection and an entering surface formed at an angle relative to the projection long axis of the at least one projection to allow for insertion of the single piece body via an axial force and resists withdrawal of the single piece body from the acetabulum.

7. The acetabular prosthesis of claim 2, wherein the at least one projection defines an external thread configured so that when the at least one projection is rotated into the acetabulum the single piece body is drawn towards and fixed within the acetabulum.

8. The acetabular prosthesis of claim 2, wherein the at least one projection defines a projection longitudinal axis formed at an angle relative to a body central axis of the body that is substantially perpendicular to the bone contacting surface of the body, wherein the projection long axis of the at least one projection intersects the central axis of the body substantially at the bone contacting surface.

9. The acetabular prosthesis of claim 1, wherein at least the concave articulation surface is substantially circular.

10. The acetabular prosthesis of claim 9, wherein the body has an external perimeter substantially equivalent to the external geometry of the articulation surface.

11. The acetabular prosthesis of claim 1, wherein the body includes an external perimeter that defines the extent of the articulation surface;
wherein the external perimeter defines a regular geometric shape, an irregular geometric shape, curved edges, straight edges, or combinations of straight edge portions and curved edge portions.

12. The acetabular prosthesis of claim 1, wherein the concave articulation surface is sized and configured to replace only a damaged region of acetabular cartilage in the acetabulum and be contained within the acetabulum.

13. An acetabular prosthesis for implanting within an acetabulum, comprising:
a single-piece unitary member formed of pyrolytic carbon, having a body and a bone connection region;
the body having an articulation surface and a bone contacting surface opposite one another on the body, the body further having an external perimeter configured to fill a selected area in an acetabular cartilage in the acetabulum;
the bone connection region operable to engage and connect to a bone structure adjacent to the defect in the acetabular cartilage.

14. The acetabular prosthesis of claim 13, wherein the body has a thickness configured to be substantially equivalent in thickness to an adjacent cartilage portion so that the articulation surface is substantially continuous to an adjacent acetabular cartilage articulation region adjacent to the selected area in the acetabular cartilage.

15. The acetabular prosthesis of claim 14, wherein the bone connection region includes at least one projection extending along a long axis from the bone contacting surface of the body and operable to be pushed into a surface of the pelvis.

16. The acetabular prosthesis of claim 15, wherein the at least one projection of the bone connection region includes one or more barbs to resist withdrawal of the single-piece member from the acetabulum.

17. The acetabular prosthesis of claim 15, wherein the at least one projection of the bone connection region includes an external edge that is angled relative to the long axis of the at least one projection and tapers to a minimal external dimension distal from the bone contacting surface of the body.

18. The acetabular prosthesis of claim 15, wherein the bone contacting surface defines a plane and the long axis of the at least one projection of the bone connection region extends at an angle relative to the plane of the bone contacting surface;
wherein the angle of the long axis of the at least one projection allows the single-piece unitary member to be positioned into the acetabulum along the long axis of the at least one projection.

19. The acetabular prosthesis of claim 18, wherein the long axis of the at least one projection intersects the plane of the bone contacting surface a distance away from a point where a line passing through a center of the articulation surface and normal to the plane of the bone contacting surface intersects the plane of the bone contacting surface.

20. The acetabular prosthesis of claim 14, wherein the external perimeter includes a curved edge, a straight edge, a combination of curved edges and straight edges, and defines a regular geometric shape or an irregular geometric shape.

21. An acetabular prosthesis for implantation within an acetabulum, comprising:
a single-piece unitary member formed of pyrolytic carbon, having a body and a bone connection region;
wherein the body has an external perimeter that defines an external extent of an articulation surface and a bone contacting surface opposite the articulation surface on the body;
wherein the external perimeter of the body is configured to fill a selected area in an acetabular cartilage in the acetabulum and contact adjacent acetabular cartilage to define a continuous articulation surface when the single-piece unitary member is placed within the acetabulum;
wherein the bone connection region is configured to engage and connect to a bone structure of the acetabulum including at least one surface feature to resist removal of the single-piece unitary member from engagement with the bone structure of the acetabulum.
